(19) United States
(12) Patent Application Publication
(10) Pub. No.: US 2017/0296247 A1
(43) Pub. Date: Oct. 19, 2017

(54) BONE CEMENT INJECTION DEVICE

(71) Applicant: Shao-Kang Hsueh, New Taipei City (TW)

(72) Inventor: Shao-Kang Hsueh, New Taipei City (TW)

(21) Appl. No.: 15/098,320

(22) Filed: Apr. 14, 2016

(51) Int. Cl. A61B 17/88; A61B 17/00

(52) U.S. Cl. CPC A61B 17/8825; A61B 2017/00907

(57) ABSTRACT

A bone cement injection device includes an outer barrel including a side slot; an injection unit movably received in the outer barrel and including an inner tube that defines a first hollow section therein and an outer tube that defmes, in combination with the inner tube, a second hollow section therebetween and includes a guide tube mounted to the outer tube to be movably received in the side slot and in communication with the second hollow section; and a pusher unit movably received in the first hollow section and positioned against a bottom end of the outer barrel. Bone cement is filled in the first hollow section and is driven by the pusher unit into a patient's bone. An air compressor is used in combination with the guide tube to induce a pressure difference between inside and outside of the bone to help injection the bone cement into the bone.

### TECHNICAL FIELD OF THE INVENTION

The present invention relates generally to innovation of bone cement injectors, and more particularly to a device usable for injection of bone cement.

### DESCRIPTION OF THE PRIOR ART

Bone cement is commonly used in orthopedic surgery, such as knee or hip replacement and vertebraplasty. Vertebaplasty is widely used in treating compression bone fractures caused by osteoporosis or bone facture and pain caused by metastatic bone tumors. To inject bone cement, an injector or injection device must be involved in order to inject bone cement into the bones of a patient.

Taiwan Utility Models M421803, which proposes a structure of bone cement injector, M 430285, which proposes an assembly of bone cement injection and driving device, and M346407, which proposes an assistive compression device of a bone cement injector, all disclose solutions of injecting bone cement into the bones of a patient; however, the structures of these prior art devices are obviously complicated. Further, to conduct injection of bone cement with the prior art devices proposed by these documents, a screw must be involved for conducting the injection, followed by an operation of rotation in order to drive the bone cement into the bones. However, since blood and air exist in the bones, due to the blood and air being present invention a closed space inside the bone, to conduct the injection of bone cement, a surgeon must apply a great effort to achieve smooth driving of the bone cement into the bone. This makes it hard to carry out the injection and the process is excessively slot. The surgeon would suffer additional and extensive stress to carry out the operation and the patient suffers increased risk of infection or pulmonary embolism.

### SUMMARY OF THE INVENTION

The primary object of the present invention is to provide a bone cement injection device that allows bone cement to be filled in a first hollow section so that through pulling an injection unit, a pusher unit drives the bone cement into a patient's bone, wherein during the process of injection, an air compressor is put into operation in combination with a guide tube to suck blood and air out of the bone through a second hollow section, whereby when the blood and air are sucked out, a pressure difference is induced to allow the bone cement to be sucked into the bone, and as such, the present invention has a simple structure, is easy to operate in an effortless manner, and may achieve an effect of efficient.

To achieve the above object, the present invention providers a bone cement injection device, which comprises: an outer barrel that comprises a side slot formed therein; an injection unit movably received in the outer barrel and comprising an inner tube that forms therein a first hollow section and an outer tube that is arranged outside and spaced from an outer surface of the inner tube by a predetermined distance to define a second hollow section between the inner tube and the outer tube, the outer tube comprising a guide tube mounted thereto to be received in the side slot and in communication with the second hollow section; and a pusher unit movably received in the first hollow section and positioned against a bottom end of the outer barrel.

In an embodiment of the present invention, the outer barrel has a top end to which a cushion section is mounted.

In an embodiment of the present invention, the outer barrel has an outer surface on which a graduation section is formed.

In an embodiment of the present invention, the outer barrel and the injection unit are made of a metallic material.

In an embodiment of the present invention, the outer barrel and the injection unit are made of a non-metallic material.

In an embodiment of the present invention, the outer barrel and the injection unit are made of a light-transmitting material.

In an embodiment of the present invention, the pusher unit comprises a pusher plunger that is movably received in the first hollow section and a base mounted to an end of the pusher plunger and positioned against the bottom end of the outer barrel.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is an exploded view of the present invention.

FIG. 3 is a cross-sectional view illustrating an operation condition of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

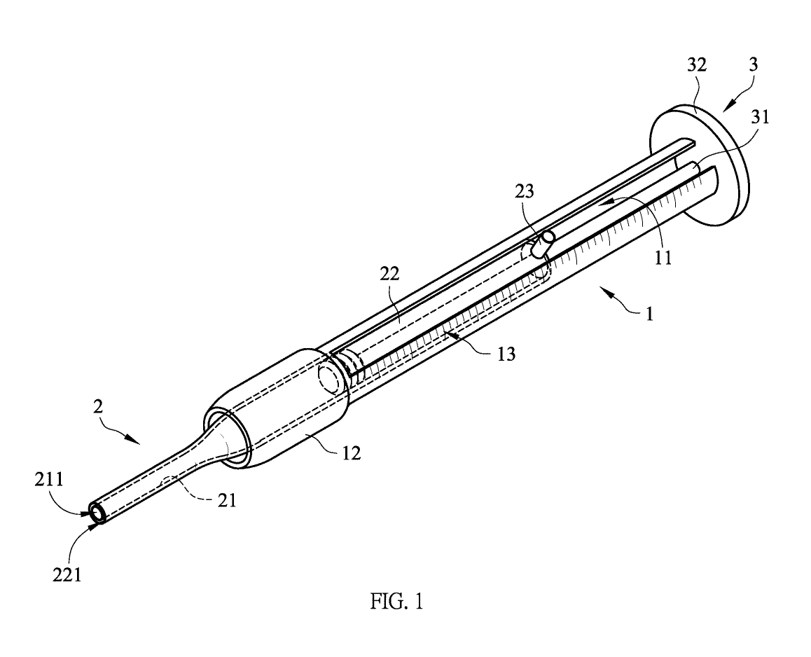

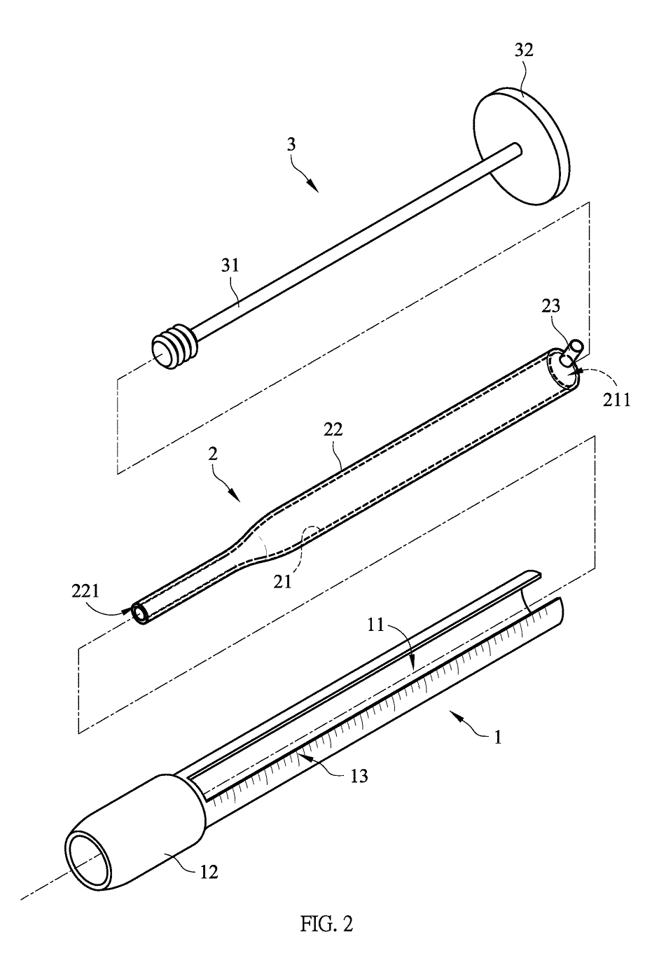

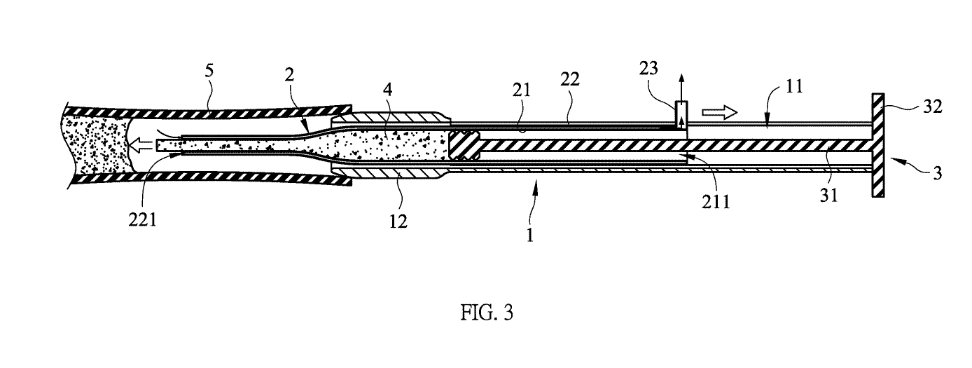

Referring to FIGS. 1-3, which are respectively a perspective view and an exploded view of the present invention and a cross-sectional view illustrating an operation of the present invention, as shown in the drawings, the present invention provides a bone cement injection device, which comprises at least an outer barrel 1, an injection unit 2, and a pusher unit 3, wherein the outer barrel 1 and the injection unit 2 are made of a metallic material, or a non-metallic material, or a light-transmitting material.

The outer barrel 1 has a circumferential wall in which a side slot 11 is formed and extending in an axial direction. The outer barrel 1 has an axial end, which is a top end thereof to which a cushion section 12 is mounted. The circumferential wall of the outer barrel 1 has an outside surface that comprises a scale section 13 having graduation markings formed thereon.

The injection unit 2 is movably received in the outer barrel 1. The injection unit 2 comprises an inner tube 21 that forms therein a first hollow section 211 and an outer tube 22 that is arranged outside and spaced from an outer surface of the inner tube 21 by a predetermined distance so that the inner tube 21 and the outer tube 22 define therebetween a second hollow section 221. The outer tube 22 comprises, mounted thereto, a guide tube 23 that is movably receivable in the side slot 11 and in communication with the second hollow section 221.

The pusher unit 3 is movably received in the first hollow section 211 and positionable against a bottom end of the outer barrel 1. The pusher unit 3 comprises a pusher plunger 31 that is movably received in the first hollow section 211 and a base 32 that is mounted to an end of the pusher plunger 31 and positioable against the bottom end of the outer barrel 1. As such, with the above-described technical features properly assembled, a novel bone cement injection device is provided.

To use the present invention, bone cement 4 is filled in the first hollow section 211 of the inner tube 2 and the guide tube 23 is connected to a tube of an air compressor (not shown). The cushion section 12 of the outer barrel 1 and an end of the injection unit 2 are inserted into a bone 5 into which the bone cement 4 is to be injected. The guide tube 23 is then pulled in a direction toward the base 32 such that the injection unit 2 is moved rearward to allow the bone cement 4 that is driven by a relative movement of the pusher plunger 31 to be discharged through an end of the first hollow section 211. At the same time when the bone cement 4 is discharged through the end of the first hollow section 211, the air compressor is activated to operate in combination with the guide tube 23 in order to allow blood and air existing in the bone 5 to be sucked out through the second hollow section 221. When the blood and air are sucked out, a pressure difference is generated between the inside and outside of the bone 5 (meaning a vacuum condition is induced inside the bone 5) so that the bone cement 4 is sucked, by the pressure difference, into the bone 5, whereby the operation of injection of the bone cement 4 is thus made easy and relatively effortless.

Further, during the injection processor a surgical operation, the graduation section 13 provided on the outer barrel 1 is used to help control the amount of the bone cement 4 injected in order to prevent over injection or under injection.

In summary, the prevent invention provides a bone cement injection device that effectively overcomes the drawbacks and shortcomings of the prior art devices and allows bone cement to be first filled in a first hollow section so that through pulling of an injection unit to allow a pusher unit to drive the bone cement into a patient's bone. During the injection, an air compressor is used in combination with a guide tube to suck blood and air out of the bone through a second hollow section so that when the blood and air are sucked out, a pressure difference is induced to help suck the bone cement into the bone. The present invention has a simple structure, is easy to operate in an effortless manner, and may achieve an effect of efficient injection.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A bone cement injection device, comprising:
an outer barrel, which comprises a side slot formed therein;
an injection unit, which is movably received in the outer barrel, the injection unit comprising an inner tube that forms therein a first hollow section and an outer tube that is arranged outside and spaced from an outer surface of the inner tube by predetermined distance such that the inner tube and the outer tube define therebetween a second hollow section, the outer tube comprising a guide tube mounted thereto and movably received in the side slot and in communication with the second hollow section; and
a pusher unit, which is movably received in the first hollow section and positioned against a bottom end of the outer barrel.

2. The bone cement injection device according to claim 1, wherein the outer barrel has a top end to which a cushion section is mounted.

3. The bone cement injection device according to claim 1, wherein the outer barrel has an outer surface on which a graduation section is formed.

4. The bone cement injection device according to claim 1, wherein the outer barrel and the injection unit are made of a metallic material.

5. The bone cement injection device according to claim 1, wherein the outer barrel and the injection unit are made of a non-metallic material.

6. The bone cement injection device according to claim 1, wherein the outer barrel and the injection unit are made of a light-transmitting material.

7. The bone cement injection device according to claim 1, wherein the pusher unit comprises a pusher plunger movably received in the first hollow section and a base mounted to an end of the pusher plunger and positioned against the bottom end of the outer barrel.
